(19) United States
(12) Patent Application Publication
(10) Pub. No.: US 2004/0115622 A1
(43) Pub. Date: Jun. 17, 2004

(54) Mixture of peptides originating from a Nef protein and applications thereof

(76) Inventors: Bernard Maillere, Versailles (FR); Sandra Pouvelle-Moratille, Saint Genevieve des Bois (FR); Jean-Gerard Guillet, Paris (FR); Hanne Gahery-Segard, Paris (FR)

Correspondence Address: MORGAN LEWIS & BOCKIUS LLP, 1111 PENNSYLVANIA AVENUE NW, WASHINGTON, DC, 20004, US

(21) Appl. No.: 10/467,322

(22) PCT Filed: Feb. 7, 2002
(86) PCT No.: PCT/FR02/00471

(30) Foreign Application Priority Data
Feb. 8, 2001 (FR) 01/01700

(51) Int. Cl. C12Q 1/70; A61K 39/21; C07K 14/16

(52) U.S. Cl. 435/5; 424/188.1; 530/350

(57) ABSTRACT

The invention relates to a mixture of peptides originating from a Nef protein and the applications thereof as a medicine (in immunogenic compositions which stimulate the production in vivo of anti-HIV T CD4+ lymphocytes and are therefore useful for vaccinating against AIDS) or as a reactive diagnosis agent for T lymphocytes particular to HIV, particularly to assess the immune status of HIV-positive patients or patients undergoing anti-retroviral therapy. Each of said peptides in the mixture is linked to at least three HLA-DR molecules encoded by the alleles selected from the group comprising alleles HLA DRB1*0101, DRB1*0301, DRB1*0401, DRB1*0701, DRB1*1101, DRB1*1301 and DRB1*1501 (molecules DR1, DR3, DR4, DR7, DR11, DR13 and DR15) and to at least one HLA-DR molecule encoded by the alleles selected from the group comprising alleles HLA DRB3*0101, DRB4*0101 and DRB5*0101 (B3, B4 and B5;), with a binding capacity of<1000 nM, said mixture of peptides linking all of the aforementioned alleles.

The present invention relates to a mixture of peptides originating from a Nef protein, and also to applications thereof as a drug (in immunogenic compositions able to stimulate the production of anti-HIV CD4+ T lymphocytes in vivo and therefore useful for vaccination against AIDS) or as a reagent for diagnosing HIV-specific T lymphocytes, in particular for evaluating the immune state of seropositive patients or patients undergoing antiretroviral therapy.

CD4+ T lymphocytes have a rearranged T receptor which allows them to selectively recognize the peptide fragments derived from degradation of the antigen by presenting cells and presented by molecules of the class II major histocompatibility complex (MHC II). The determinants borne by these peptide fragments and effectively recognized by T lymphocytes are called T epitopes.

CD4+ T lymphocytes are known to be a preferred target of the human immunodeficiency virus (HIV) and can go as far as to disappear completely from the blood circulation.

As a result, patients infected with HIV suffer from opportunistic infections and cannot induce vigorous immune responses to combat them.

CD4+ T lymphocytes in fact have a major role in establishing immune responses. They secrete most of the cytokines required to recruit effector cells, namely cytotoxic CD8+ T lymphocytes and antibody-producing B lymphocytes. They are also involved in activating cells by cellular contacts and, for example, induce the activation, via CD40, of antigen-presenting dendritic cells.

By virtue of their major role, the disappearance of the CD4+ T lymphocytes therefore leads to a significant weakening of the immune system: in particular, the disappearance of the CD4+ T lymphocytes decreases the specific immunity against HIV. In fact, recent studies have shown that individuals who exhibit a proliferative response specific for HIV-component-specific CD4+ cells can control the viral infection in the absence of any treatment (1, 2).

A reverse correlation is even noted between the proliferation of CD4+ T lymphocytes with respect to the P24 protein, and viral load. It is possible that this correlation results directly from the ability of the TH1-type CD4+ T lymphocytes to secrete antiviral cytokines such as γ-IFN, and to be cytotoxic. It may also result indirectly from their ability to maintain humoral immunity, and more probably cellular immunity. It is in fact known that cytotoxic CD8+ T cells contribute considerably to maintaining seropositive patients in a nonsymptomatic state.

The recent progress made with anti-HIV chemo-therapy allow to hope that it may be possible to re-establish normal and protective immunity in patients infected with this virus. It is in fact observed that, in patients undergoing highly active antiretroviral therapy (HAART), the level of CD4+ cells increases considerably (3). This increase in the number of CD4+ T lymphocytes is a necessary condition for the recruitment of CD4+ T lymphocytes, but it rarely allow to obtain a proliferative response against components of the virus. Induction of this response in fact requires a specific treatment, such as a vaccine.

The activation of CD4+ T lymphocytes occurs under the effect of the presentation of the viral peptides by HLA II molecules, borne by the antigen-presenting cells (APCs). These peptides, called T epitopes, are the result of proteolytic degradation of the viral antigens by the APC. They are variable in length, generally from 13 to 25 amino acids, and have a sequence which makes them capable of binding to HLA II molecules.

It is now established that a peptide, T epitope, is capable, to the same degree as the native antigen, of stimulating, in vitro, CD4+ T lymphocytes which are specific for it, or of recruiting them in vivo.

T epitopes are therefore sufficient to induce a CD4+ response. However, one of the major problems which limits the use of these peptides is that their sequence varies from one individual to another, due to the polymorphism of the HLA II molecules, which are hetero-dimers, expressed on the antigen-presenting cells (APCs) and which present to the CD4+ T lymphocytes the T epitopes of said antigens. These molecules are capable of binding a large repertoire of peptides having very different sequences, which allows them to present to the T cells several peptides per antigen.

Four different types of HLA II molecule exist per individual: 2 HLA-DR, 1 HLA-DQ and 1 HLA-DP; the HLA-DR molecule in which the β chain is encoded by the DRB1 gene (1st gene) is the most commonly expressed. Currently, more than 200 different alleles are listed for DRB1, which alleles define various antigens or types as summarized in table I below.

TABLE I
Molecules expressed by various HLA-DRB1 alleles

| Antigen | Allele | Alias |
|---|---|---|
| DR1 | DRB1*0101 | DR1 |
| DR3 | DRB1*0301 | DR3w17 |
| DR4 | DRB1*0401 | DR4w4 |
| | DRB1*0405 | DR4w15 |
| DR7 | DRB1*0701 | DR7 |
| DR8 | DRB1*0802 | DR8w2 |
| DR9 | DRB1*0901 | DR9 |
| DR11 | DRB1*1101 | DR5w11 |
| DR12 | DRB1*1201 | DR5w12 |
| DR13 | DRB1*1301 | |
| | DRB1*1302 | DR6w19 |
| DR15 | DRB1*1501 | DR2w2b |

Each allele possesses its own binding properties; the broad specificity of the HLA II molecules and the existence of several isoforms and of a polymorphism means that each individual recognizes, in an antigen, a set of peptides the nature of which depends on the HLA II molecules which characterize it. Since a large number of HLA II alleles exist, a large number of T epitopes, specific to each allele, therefore exist for a given antigen. The distribution of the alleles in a given population is not homogeneous; for example, in the French population, which corresponds to a mainly Caucasian population, only 7 alleles of the DRB1 locus exceed 5%; these are the alleles: DRB1*0101, DRB1*0301, DRB1*0401, DRB1*0701, DRB1*1101, DRB1*1301 and DRB1*1501, which represent 64% of the population (4). These same alleles are also in the majority in other European populations, where their frequency ranges from 53% (Spain) to 82% (Denmark)^{t}, and also in North America (55-58%).

The HLA-DRB3, -DRB4 and -DRB5 molecules (2nd gene), which are HLA-DR molecules in which the β chain is not encoded by the DRB1 gene, are also present with considerable allelic frequencies in the various Caucasian populations: 9.2% for DRB3*0101 (B3), 28.4% for DRB4*0101 (B4) and 7.9% for DRB5*0101 (B5). They therefore cover, on their own, 45% of the allelic frequency of Caucasian populations.

The peptides present in a peptide sequence and which bind all of these alleles include the T epitopes of the majority of the Caucasian population.

With regard to HIV:

most of the epitopes already described are HLA I molecule-restricted [(5), (6), international application WO 98/50423, international application WO 99/27954, international application WO 99/40113, international application WO 99/51630, international application WO 00/75181]. They are therefore peptides recognized by CD8+ T lymphocytes. HLA I molecules (HLA-A, -B or -C) have binding characteristics which are totally different from class II molecules. They bind shorter peptides, in principle of 9 amino acids, and the binding motifs are specific to each allele. For HIV, HLA I molecule-restricted sequences have been found in the Env, Pol, Gag and Nef proteins, the last two being the most commonly recognized (7).

For example, Gahéry-Ségard et al. [(11) and international application WO 99/27954] have described peptides derivated from the Nef protein (peptides corresponding respectively to amino acids 66-97 (N1), 117-147 (N2) and 182-205 (N3) of the Nef protein) which comprise HLA I molecule-restricted T epitopes; more precisely, the T epitopes demonstrated in said N1, N2 and N3 peptides are as follows:

TABLE II

| Nef protein positions | HLA I restriction |
|---|---|
| 121-128, 137-145, 184-191 and | HLA-A1 |
| 195-202 | |
| 136-145, 190-198 | HLA-A2 |
| 73-82, 84-92 | HLA-A11 |
| 90-97, 182-189 | HLA-B8 |
| 134-141 | HLA-B27 |
| 135-143 | HLA-B18 |

To produce an anti-HIV vaccine, Gahéry-Ségard et al. (11) recommend combining said N1, N2 and N3 peptides with two peptides derivated from the Gag protein (G1 and G2) and with a peptide derivated from the Env protein (E); all these peptides are modified at their C-terminal end by the addition of a palmitoyl lysylamide group [table 1 of (11)]. The immunogenicity and the tolerance of this lipopeptide vaccine were evaluated in seronegative volunteers. The specific T response is evaluated using a T cell proliferation assay. The N1 peptide induces T cell proliferation in 5/10 of the vaccinated donors, the N2 peptide induces proliferation only in a single vaccinated donor, and the N3 peptide induces proliferation in 4/10 donors. Although a CD4+ T response is observed, it is very random and variable with the N1, N2 and N3 peptides.

HLA II molecule-restricted peptides have also been described; these are peptide sequences (T epitopes) derivated from the Nef protein (10), and more particularly the Nef peptides 13-23, 46-53, 44-81, 69-82, 180-193 and 194-210 (table 4 of 10) which were identified using T clones specific for the Nef protein; the variation in the peptides affects in an essential manner the T cell response to the Nef protein. The abovementioned peptides are restricted with respect to the following HLA II molecules [table 7 of (10)]:

TABLE III

| | | HLA IIs which restrict |
|---|---|---|
| | Peptides | presentation of the peptides to |
| T clones | recognized | said clones |
| MM Nef 95.12 | 13-23 | DRw6 |
| MM Nef 33.6 | 13-23 | DRw6 |
| JB Nef 1.13 | 46-53 | DQw7 |
| SP Nef 29.16 | 69-82 | DR1, DRw15(2) (=DRB1*1501) |
| GK Nef 6.38 | 180-193 | DP5 |
| JB Nef 3.2 | 194-210 | DR1 |
| DS Nef 59.25 | 44-81 | DRw15(2) (=DRB1*1501) |

All of the peptides proposed until now correspond to T epitopes, selected for their ability to be conserved among the various isolates; however, they are specific only for particular individuals; in fact, an interindividual variability of the T epitopes exists, which makes the choice of molecules suitable for mass vaccination against AIDS difficult; consequently, the peptides described above are not suitable for preparing an immunogenic and vaccine composition able to stimulate anti-HIV CD4+ T lymphocytes and to generate a protective immune response, irrespective of the individual to be protected, since they do not stimulate a protective CD4+ T response in all individuals to be treated.

For this reason, the inventors have given themselves the aim of providing a set of peptides able to be incorporated into an immunogenic composition and to stimulate anti-HIV CD4+ T lymphocytes in most European or North American Caucasian individuals, which is particularly advantageous in combination with highly active antiretroviral therapy (tritherapy for example), so as to effectively induce a specific proliferative response against components of the virus.

Such a set has the property of being effective in a large number of individuals, whereas the peptides of the prior art are active in some individuals and are inactive in the majority of other individuals because the latter do not recognize the Nef protein via the same determinants.

To do this, the inventors have selected peptides derivated from the HIV Nef protein, which are restricted with respect to the HLA II molecules predominant in Caucasian populations, and have found that, in combination, the selected peptides effectively induce an immunogenic and protective response in a large number of individuals.

Consequently, a subject of the present invention is a mixture of peptides derivated from an HIV Nef protein, characterized in that each one of said peptides binds to at least three HLA-DR molecules encoded by the alleles selected from the group consisting of the HLA alleles DRB1*0101, DRB1*0301, DRB1*0401, DRB1*0701, DRB1*1101, DRB1*1301 and DRB1*1501 (molecules DR1, DR3, DR4, DR7, DR11, DR13 and DR15) and to at least one HLA-DR molecule encoded by the alleles selected from the group consisting of the HLA alleles DRB3*0101, DRB4*0101 and DRB5*0101 (B3, B4 and B5), with a binding activity<1 000 nM, said mixture of peptides binding all the abovementioned alleles.

Such a mixture of peptides makes it possible to obtain, surprisingly, a proliferative CD4+ T response (stimulation of CD4+ T lymphocytes) and also a stimulation of the CTL response, in the vast majority of the Caucasian population to be protected; it can therefore be considered that such a mixture constitutes a first step toward an “universal” immunogenic composition which can be used in a vaccine.

According to an advantageous embodiment of said mixture, said peptides originate from the Nef protein of any HIV-1 strain.

According to another advantageous embodiment of said mixture, said peptides are selected from the group consisting of the peptide corresponding to positions 1-36 (Nef1 peptide), the peptide corresponding to positions 66-94 (Nef4 peptide), the peptide corresponding to positions 74-88 (Nef4.4 peptide), the peptide corresponding to positions 77-91 (Nef4.5 peptide), the peptide corresponding to positions 137-168 (Nef10 peptide), the peptide corresponding to positions 139-153 (Nef10.2 peptide), the peptide corresponding to positions 175-190 (Nef12 peptide), the peptide corresponding to positions 182-198 (Nef13 peptide), the peptide corresponding to positions 178-192 (Nef13.1 peptide), the peptide corresponding to positions 181-195 (Nef13.2 peptide), the peptide corresponding to positions 183-197 (Nef13.3 peptide), the peptide corresponding to positions 187-201 (Nef13.4 peptide) and the peptide corresponding to positions 189-203 (Nef13.5 peptide) of the Nef protein, with reference to the Bru strain (13).

Particularly advantageously, said mixture of peptides according to the invention is selected from the group consisting of the following mixtures, in which the positions of the peptides are also specified with references to the positions of the Nef protein of the Bru strain (13):

a mixture comprising: the peptide corresponding to positions 66-94 (Nef4), the peptide corresponding to positions 182-198 (Nef13);

a mixture comprising: the peptide corresponding to positions 66-94 (Nef4), the peptide corresponding to positions 178-192 (Nef13.1);

a mixture comprising: the peptide corresponding to positions 66-94 (Nef4), the peptide corresponding to positions 181-195 (Nef13.2);

a mixture comprising: the peptide corresponding to positions 74-88 (Nef4.4), the peptide corresponding to positions 182-198 (Nef13);

a mixture comprising: the peptide corresponding to positions 74-88 (Nef4.4), the peptide corresponding to positions 178-192 (Nef13.1);

a mixture comprising: the peptide corresponding to positions 74-88 (Nef4.4), the peptide corresponding to positions 181-195 (Nef13.2);

a mixture comprising: the peptide corresponding to positions 1-36 (Nef1), the peptide corresponding to positions 66-94 (Nef4) and the peptide corresponding to positions 182-198 (Nef13);

a mixture comprising: the peptide corresponding to positions 1-36 (Nef1), the peptide corresponding to positions 74-88 (Nef4.4) and the peptide corresponding to positions 182-198 (Nef13);

a mixture comprising: the peptide corresponding to positions 1-36 (Nef1), the peptide corresponding to positions 77-91 (Nef4.5) and the peptide corresponding to positions 182-198 (Nef13);

a mixture comprising: the peptide corresponding to positions 1-36 (Nef1), the peptide corresponding to positions 66-94 (Nef4) and the peptide corresponding to positions 178-192 (Nef13.1);

a mixture comprising: the peptide corresponding to positions 1-36 (Nef1), the peptide corresponding to positions 74-88 (Nef4.4) and the peptide corresponding to positions 178-192 (Nef13.1);

a mixture comprising: the peptide corresponding to positions 1-36 (Nef1), the peptide corresponding to positions 77-91 (Nef4.5) and the peptide corresponding to positions 178-192 (Nef13.1);

a mixture comprising: the peptide corresponding to positions 1-36 (Nef1), the peptide corresponding to positions 66-94 (Nef4) and the peptide corresponding to positions 181-195 (Nef13.2);

a mixture comprising: the peptide corresponding to positions 1-36 (Nef1), the peptide corresponding to positions 74-88 (Nef4.4) and the peptide corresponding to positions 181-195 (Nef13.2);

a mixture comprising: the peptide corresponding to positions 1-36 (Nef1), the peptide corresponding to positions 77-91 (Nef4.5) and the peptide corresponding to positions 181-195 (Nef13.2);

a mixture comprising: the peptide corresponding to positions 1-36 (Nef1), the peptide corresponding to positions 137-168 (Nef10) and the peptide corresponding to positions 175-190 (Nef12);

a mixture comprising: the peptide corresponding to positions 1-36 (Nef1), the peptide corresponding to positions 137-168 (Nef10) and the peptide corresponding to positions 182-198 (Nef13);

a mixture comprising: the peptide corresponding to positions 1-36 (Nef1), the peptide corresponding to positions 137-168 (Nef10) and the peptide corresponding to positions 178-192 (Nef13.1);

a mixture comprising: the peptide corresponding to positions 1-36 (Nef1), the peptide corresponding to positions 137-168 (Nef10) and the peptide corresponding to positions 181-195 (Nef13.2);

a mixture comprising: the peptide corresponding to positions 1-36 (Nef1), the peptide corresponding to positions 139-153 (Nef10.2) and the peptide corresponding to positions 182-198 (Nef13);

a mixture comprising: the peptide corresponding to positions 1-36 (Nef1), the peptide corresponding to positions 139-153 (Nef10.2) and the peptide corresponding to positions 178-192 (Nef13.1);

a mixture comprising: the peptide corresponding to positions 1-36 (Nef1), the peptide corresponding to positions 139-153 (Nef10.2) and the peptide corresponding to positions 181-195 (Nef13.2);

a mixture comprising: the peptide corresponding to positions 1-36 (Nef1), the peptide corresponding to positions 175-190 (Nef12) and the peptide corresponding to positions 182-198 (Nef13);

a mixture comprising: the peptide corresponding to positions 1-36 (Nef1), the peptide corresponding to positions 175-190 (Nef12) and the peptide corresponding to positions 178-192 (Nef13.1);

a mixture comprising: the peptide corresponding to positions 1-36 (Nef1), the peptide corresponding to positions 175-190 (Nef12) and the peptide corresponding to positions 181-195 (Nef13.2);

a mixture comprising: the peptide corresponding to positions 66-94 (Nef4), the peptide corresponding to positions 137-168 (Nef10) and the peptide corresponding to positions 182-198 (Nef13);

a mixture comprising: the peptide corresponding to positions 66-94 (Nef4), the peptide corresponding to positions 137-168 (Nef10) and the peptide corresponding to positions 178-192 (Nef13.1);

a mixture comprising: the peptide corresponding to positions 66-94 (Nef4), the peptide corresponding to positions 137-168 (Nef10) and the peptide corresponding to positions 181-195 (Nef13.2);

a mixture comprising: the peptide corresponding to positions 66-94 (Nef4), the peptide corresponding to positions 139-153 (Nef10.2) and the peptide corresponding to positions 182-198 (Nef13);

a mixture comprising: the peptide corresponding to positions 66-94 (Nef4), the peptide corresponding to positions 139-153 (Nef10.2) and the peptide corresponding to positions 178-192 (Nef13.1);

a mixture comprising: the peptide corresponding to positions 66-94 (Nef4), the peptide corresponding to positions 139-153 (Nef10.2) and the peptide corresponding to positions 181-195 (Nef13.2);

a mixture comprising: the peptide corresponding to positions 66-94 (Nef4), the peptide corresponding to positions 175-190 (Nef12) and the peptide corresponding to positions 182-198 (Nef13);

a mixture comprising: the peptide corresponding to positions 66-94 (Nef4), the peptide corresponding to positions 175-190 (Nef12) and the peptide corresponding to positions 178-192 (Nef13.1);

a mixture comprising: the peptide corresponding to positions 66-94 (Nef4), the peptide corresponding to positions 175-190 (Nef12) and the peptide corresponding to positions 181-195 (Nef13.2);

a mixture comprising: the peptide corresponding to positions 66-94 (Nef4), the peptide corresponding to positions 175-190 (Nef12) and the peptide corresponding to positions 183-197 (Nef13.3);

a mixture comprising: the peptide corresponding to positions 66-94 (Nef4), the peptide corresponding to positions 175-190 (Nef12) and the peptide corresponding to positions 187-201 (Nef13.4);

a mixture comprising: the peptide corresponding to positions 66-94 (Nef4), the peptide corresponding to positions 175-190 (Nef12) and the peptide corresponding to positions 189-203 (Nef13.5);

a mixture comprising: the peptide corresponding to positions 74-88 (Nef4.4), the peptide corresponding to positions 137-168 (Nef10) and the peptide corresponding to positions 182-198 (Nef13);

a mixture comprising: the peptide corresponding to positions 74-88 (Nef4.4), the peptide corresponding to positions 137-168 (Nef10) and the peptide corresponding to positions 178-192 (Nef13.1);

a mixture comprising: the peptide corresponding to positions 74-88 (Nef4.4), the peptide corresponding to positions 137-168 (Nef10) and the peptide corresponding to positions 181-195 (Nef13.2);

a mixture comprising: the peptide corresponding to positions 74-88 (Nef4.4), the peptide corresponding to positions 175-190 (Nef12) and the peptide corresponding to positions 182-198 (Nef13);

a mixture comprising: the peptide corresponding to positions 74-88 (Nef4.4), the peptide corresponding to positions 175-190 (Nef12) and the peptide corresponding to positions 178-192 (Nef13.1);

a mixture comprising: the peptide corresponding to positions 74-88 (Nef4.4), the peptide corresponding to positions 175-190 (Nef12) and the peptide corresponding to positions 181-195 (Nef13.2);

a mixture comprising: the peptide corresponding to positions 74-88 (Nef4.4), the peptide corresponding to positions 175-190 (Nef12) and the peptide corresponding to positions 183-197 (Nef13.3);

a mixture comprising: the peptide corresponding to positions 77-91 (Nef4.5), the peptide corresponding to positions 175-190 (Nef12) and the peptide corresponding to positions 182-198 (Nef13);

a mixture comprising: the peptide corresponding to positions 77-91 (Nef4.5), the peptide corresponding to positions 175-190 (Nef12) and the peptide corresponding to positions 178-192 (Nef13.1);

a mixture comprising: the peptide corresponding to positions 77-91 (Nef4.5), the peptide corresponding to positions 175-190 (Nef12) and the peptide corresponding to positions 181-195 (Nef13.2);

a mixture comprising: the peptide corresponding to positions 77-91 (Nef4.5), the peptide corresponding to positions 175-190 (Nef12) and the peptide corresponding to positions 183-197 (Nef13.3);

a mixture comprising: the peptide corresponding to positions 137-168 (Nef10), the peptide corresponding to positions 175-190 (Nef12) and the peptide corresponding to positions 182-198 (Nef13);

a mixture comprising: the peptide corresponding to positions 137-168 (Nef10), the peptide corresponding to positions 175-190 (Nef12) and the peptide corresponding to positions 178-192 (Nef13.1);

a mixture comprising: the peptide corresponding to positions 137-168 (Nef10), the peptide corresponding to positions 175-190 (Nef12) and the peptide corresponding to positions 181-195 (Nef13.2);

a mixture comprising: the peptide corresponding to positions 137-168 (Nef10), the peptide corresponding to positions 175-190 (Nef12) and the peptide corresponding to positions 183-197 (Nef13.3);

a mixture comprising: the peptide corresponding to positions 137-168 (Nef10), the peptide corresponding to positions 175-190 (Nef12) and the peptide corresponding to positions 187-201 (Nef13.4);

a mixture comprising: the peptide corresponding to positions 137-168 (Nef10), the peptide corresponding to positions 175-190 (Nef12) and the peptide corresponding to positions 189-203 (Nef13.5).

Specifically:

Nef1 binds with good affinity to the molecules DRB1*0101, 0401, 0701, 1101, 1301 and 1501, and DRB5*0101,

Nef4 binds with good affinity to the molecules DRB1*0101, 0401, 1101 and 1501, DRB5*0101 and DRB4*0101,

Nef4.1 binds with good affinity to the molecules DRB1*0101, 0401, 1101 and 1501, DRB5*0101 and DRB4*0101,

Nef4.2 binds with good affinity to the molecules DRB1*0101, 0401, 1101 and 1501, DRB5*0101 and DRB4*0101,

Nef10 binds with good affinity to the molecules DRB1*0101, 0301, 0401, 0701 and 1101, DRB5*0101 and DRB4*0101,

Nef10.1 binds with good affinity to the molecules DRB1*0101, 0301, 0401, 0701 and 1101, DRB5*0101 and DRB4*0101,

Nef12 binds with good affinity to the molecules DRB1*0701, 1101 and 1501, DRB3*0101 and DRB4*0101,

Nef13 binds with good affinity to the molecules DRB1*0301, 0701, 1101 and 1301, DRB3*0101 and DRB5*0101,

Nef13.1 binds with good affinity to the molecules DRB1*0301, 0701, 1101 and 1301, DRB3*0101 and DRB5*0101,

Nef13.2 binds with good affinity to the molecules DRB1*0301, 0701, 1101 and 1301, DRB3*0101 and DRB5*0101,

Nef13.3 binds with good affinity to the molecules DRB1*0301, 0701, 1101 and 1301, and DRB5*0101,

Nef13.4 binds with good affinity to the molecules DRB1*0701, 1101 and 1301, and DRB5*0101,

Nef13.5 binds with good affinity to the molecules DRB1*0701, 1101 and 1301, and DRB5*0101.

A subject of the present invention is also an anti-HIV immunogenic composition, characterized in that it comprises at least one mixture of peptides as defined above, combined with at least one pharmaceutically acceptable vehicle and, optionally, with at least one adjuvant.

The adjuvants used are adjuvants conventionally used in vaccine compositions, such as alumina hydroxide or squalene.

According to an advantageous embodiment of said immunogenic composition, said peptides are either in the form of lipopeptides, or are incorporated into a recombinant virus, a viral vector for gene therapy (adenovirus, etc.), or are included in a protein, and in particular a recombinant protein (Leclerc C. et al., Int. Rev. Immunol., 1994, 11, 2, 123-132; Janssen R. et al., Int. Rev. Immunol., 1994, 11, 2, 113-121), or are chemically modified. In the latter case, they comprise, for example, unnatural modifications such as D amino acids, pseudopeptide bonds or modifications of the C- or N-terminal ends.

The lipid component of the lipopeptide is in particular obtained by adding a lipid motif onto an α-amino function of said peptides or onto a reactive function of the side chain of an amino acid of the peptide component; it may comprise one or more C₄₋₂₀ fatty acid-derived chains, which are optionally branched or unsaturated (palmitic acid, oleic acid, linoleic acid, linolenic acid, 2-aminohexadecanoic acid, pimelautide, trimexautide) or a derivative of a steroid. The method for preparing such lipopeptides is in particular described in international applications WO 99/40113 or WO 99/51630. The preferred lipid component is in particular represented by an N^{α}-acetyl-lysine N^{ε}(palmitoyl) group, also called Ac-K(Pam).

According to another advantageous embodiment of said immunogenic composition, said mixture of peptides is combined:

with one or more peptides or lipopeptides containing one or more CD8+ epitopes (recognized specifically by cytotoxic T lymphocytes and presented by HLA I molecules), and more particularly the CD8+ epitopes derivated from a HIV-1 protein (see table II), and/or

with other peptides containing a CD4+ epitope, which peptides bind at least to one HLA-DR molecule encoded by the alleles selected from the group consisting of the HLA alleles DRB1*0101, DRB1*0301, DRB1*0401, DRB1*0701, DRB1*1101, DRB1*1301 and DRB1*1501 (molecules DR1, DR3, DR4, DR7, DR11, DR13 and DR15) or to at least one HLA-DR molecule encoded by the alleles selected from the group consisting of the HLA alleles DRB3*0101, DRB4*0101 and DRB5*0101 (B3, B4 and B5), with a binding activity<1 000 nM, such as the peptides corresponding to positions 37-71 (Nef3), to positions 113-128 (Nef7), to positions 117-132 (Nef8), to positions 132-147 (Nef9) or to positions 155-185 (Nef11) of the HIV-1 Nef protein, and/or

with other peptides comprising multiple CD4+ epitopes, such as the peptide of tetanus toxin TT (positions 830-846), the peptide of Influenza hemagglutinin HA (positions 307-319), PADRE (Pan DR Epitope, Alexandre J. et al., Immunity, 1994, 1, 9, 751-761), HIV-1 Nef peptide 45-69 and the LSA3 peptide of Plasmodium falciparum, and/or

with one or more peptides or lipopeptides containing one or more B epitopes, more particularly B epitopes derivated from a HIV-1 protein, recognized specifically by antibodies directed against said epitopes.

The Nef peptides according to the invention, included in said mixture, were advantageously selected using an HLA-DR/peptide binding assay comprising:

purifying the HLA-DR molecules of interest, i.e. those involving more than 5% of a given population, and in particular the HLA DR1, DR3, DR4, DR7, DR11, DR13 and DR15 molecules,

incubating the HLA-DR molecules thus purified, with various concentrations of overlapping fragments entirely covering the sequence of the Nef protein and with a reagent R1 or tracer consisting of a peptide fragment combined with a nonradioactive label, such as biotin, the sequence of which is different from said peptides; the reagent R1 or tracer is chosen such that it exhibits affinity with respect to one of the HLA-DR molecules of interest, such that it may be used at a concentration<200 nM,

transferring the complexes obtained onto an ELISA-type plate precoated with an antibody specific for all HLA-DRs,

revealing the HLA-DR molecule/reagent R1 complexes attached to the bottom of the plate by means of suitable conjugates, such as streptavidin-phosphatase, and of a fluorescent substrate,

selecting the peptides comprising different epitopes, i.e. the most representative of the various areas of interaction between the Nef protein and the HLA-DR molecules, and

choosing the most suitable peptides as a function of the frequency of the alleles with respect to which they exhibit a binding activity<1 000 nM, corresponding to the concentration of these peptides which inhibits 50% of the binding of the reagent R1 (IC₅₀) .

These assays make it possible to unambiguously associate with each allele of the 1st gene or of the 2nd gene the sequences of the fragments capable of binding thereto or, on the contrary, which do not bind thereto.

This approach makes it possible to define immunogenic compositions which include peptides which bind to the greatest number of different HLA-DR molecules and which can thus advantageously be protective for the majority of patients, even if their HLA molecules are not known.

This approach also has the advantage of making it possible to select peptides which are significantly more specific for HIV-1 than the approaches seeking to select peptides on the basis of their ability to stimulate CD4+ T lymphocytes.

The incubation conditions are specific for each HLA-DR molecule (incubation time, pH, reagent R1, concentration of HLA-DR or of peptide).

The reagent R1 is selected from the group consisting of the following sequences:

PKYVKQNTLKLAT (HA 306-318) (SEQ ID NO. 1), specific for the alleles DRB1*0101, DRB1*0401 and DRB1*1101,

EAEQLRAYLDGTGVE (A3 152-166) (SEQ ID NO. 2), specific for the allele DRB1*1501,

AKTIAYDEEARGLE (MT 2-16) (SEQ ID NO. 3), specific for the allele DRB1*0301,

AAYAAAKAAALAA (YKL) (SEQ ID NO. 4), specific for the allele DRB1*0701,

TERVRLVTRHIYNREE (B1 21-36) (SEQ ID NO. 5), specific for the allele DRB1*1301,

ESWGAVWRIDTPDKLTGPFT (LOL 191-210) (SEQ ID NO. 6), specific for the allele DRB3*0101, and

AGDLLAIETDKATI (E2/E168) (SEQ ID NO. 7), specific for the allele DRB4*0101.

Other reagents R1 can be used, in particular those described in Southwood et al. (14).

As a variant, said immunogenic composition advantageously comprises the sequences encoding the peptides as defined above.

In fact, the use of naked DNA for immunization constitutes an effective vaccine approach; it consists in injecting into the host organism to be vaccinated a naked DNA encoding a protein antigen; this DNA enables sustained synthesis of the antigen by the host's cells and also long-lasting presentation of this antigen to the immune system.

A subject of the present invention is also a vaccine, characterized in that it includes an immunogenic composition as defined above.

A subject of the present invention is also peptides derivated from a HIV Nef protein, characterized:

in that they contain a CD4+ epitope capable of having a binding activity<1 000 nM with respect to at least one HLA II molecule (HLA-DR) which is predominant in Caucasian populations (1st gene and/or 2nd gene), as defined above, of being recognized by CD4+ T lymphocytes specific for said peptides, and of stimulating CD4+ T lymphocytes specific for said peptides, and

in that they are selected from the group consisting of the following fragments of the Nef protein, with reference to the Bru strain: the fragment corresponding to positions 1-36 of the Nef protein (Nef1), the fragment corresponding to positions 3-17 (Nef1.1), the fragment corresponding to positions 8-22 (Nef1.2), the fragment corresponding to positions 11-25 (Nef1.3), the fragment corresponding to positions 14-28 (Nef1.4), the fragment corresponding to positions 37-71 of the Nef protein (Nef3), the fragment corresponding to positions 66-94 (Nef4), the fragment corresponding to positions 68-82 (Nef4.1), the fragment corresponding to positions 74-88 (Nef4.4), the fragment corresponding to positions 77-91 (Nef4.5), the fragment corresponding to positions 79-93 (Nef4.6), the fragment corresponding to positions 83-97 (Nef4.7), the fragment corresponding to positions 113-128 (Nef7), the fragment corresponding to positions 117-132 (Nef8), the fragment corresponding to positions 132-147 (Nef9), the fragment corresponding to positions 137-168 (Nef10), the fragment corresponding to positions 137-151 (Nef10.1), the fragment corresponding to positions 139-153 (Nef10.2), the fragment corresponding to positions 141-155 (Nef10.3), the fragment corresponding to positions 146-160 (Nef10.5), the fragment corresponding to positions 155-185 (Nef11), the fragment corresponding to positions 175-190 (Nef12), the fragment corresponding to positions 182-198 (Nef13), the fragment corresponding to positions 178-192 (Nef13.1), the fragment corresponding to positions 181-195 (Nef13.2), the fragment corresponding to positions 183-197 (Nef13.3), the fragment corresponding to positions 187-201 (Nef13.4), the fragment corresponding to positions 189-203 (Nef13.5), and the fragment corresponding to positions 191-205 (Nef13.6).

Such peptides have the advantage of binding to at least one HLA-DR molecule encoded by the HLA alleles DRB1*0101, DRB1*0301, DRB1*0401, DRB1*0701, DRB1*1101, DRB1*1301 and DRB1*1501 (molecules DR1, DR3, DR4, DR7, DR11, DR13 and DR15), and/or to at least one HLA-DR molecule encoded by the HLA alleles DRB3*0101, DRB4*0101 and DRB5*0101 (B3, B4 and B5), with a binding activity<1 000 nM.

More precisely, and in accordance with table VII below:

the Nef1.2, Nef1.3, Nef1.4, Nef3, Nef4.2, Nef4.6, Nef4.7, Nef9, Nef10.5, Nef11 and Nef13.6 peptides bind to less than three HLA-DRB1 molecules, with a binding activity<1 000 nM, and

the Nef1, Nef4, Nef4.4, Nef4.5, Nef10, Nef10.2, Nef12, Nef13, Nef13.1, Nef13.2, Nef13.3, Nef13.4 and Nef13.5 peptides bind to at least three HLA-DRB1 molecules and to at least one HLA-DR molecule encoded by a DRB3, DRB4 or DRB5 allele, with a binding activity<1 000 nM.

Such binding activities make it possible to use said peptides:

either as a mixture, under the conditions defined above, in immunogenic compositions, insofar as they are capable of inducing a proliferative response of the CD4+ T cells and therefore of helping to induce a cytotoxic or humoral response in the majority of Caucasian populations. They can therefore advantageously be incorporated into a vaccine composition;

or separately, as a diagnostic reagent, preferably in the form of tetramers, for evaluating the immune state of a healthy individual or of seropositive patients for HIV or suffering from AIDS. For example, by virtue of these various peptides, it is possible to detect the presence of Nef-specific CD4+ T cells with proliferation or ELISPOT assays (qualitative evaluation), or to carry out cell sorting by flow cytometry, using the peptides which have been selected (quantitative evaluation) and labeled: for example, by flow cytometry, it is possible to evaluate the level of Nef protein-specific cells relative to the blood cell population.

Consequently, a subject of the present invention is also a diagnostic reagent, characterized in that it is selected from the group consisting of a mixture of peptides as defined above, or one of the peptides also as defined above, said peptides being optionally labelled or complexed, in the form of multimeric complexes.

Preferably, said diagnostic reagent consists of the mixtures of peptides obtained from the Nef1, Nef4, Nef10, Nef12 and Nef13 peptides.

A subject of the present invention is also a method for evaluating the immune state of an individual, characterized in that it comprises a step of detecting the presence of CD4+ T cells specific for the Nef peptides as defined above; said detection is advantageously carried out using one of the following assays: proliferation assay, ELISPOT assay [see, for example, international application WO 99/51630 or Gahéry-Ségard et al. (11)] or flow cytometry in the presence of multimeric complexes constituted from said Nef peptides.

More precisely:

as regards the proliferation assay:

A suspension of cells (PBMCs, PBMCs depleted of CD8+ cells, T lymphocytes pre-enriched via a step of culturing in vitro with the peptides selected according to the invention or cloned T lymphocytes) is cultured for 3 to 5 days in the presence of the selected peptides and as required of suitable presenting cells, such as dendritic cells, autologous or heterologous PBMCs, lymphoblastoid cells such as those obtained after infection with the EBV virus or genetically modified cells. The proliferation of the cells is measured by incorporation of tritiated thymidine into the DNA of the cells. The peptides selected in accordance with the invention make it possible to reveal, in the initial suspension, the presence of cells specific for these peptides.

as regards the ELISPOT assay:

The ELISPOT assay makes it possible to reveal the presence of T cells specific for a peptide selected in accordance with the invention and secreting γ-IFN.

More precisely, the T cells are revealed by measuring the secretion of γ-IFN after incubation of the PMBCs from patients, with the peptides selected according to the invention, in accordance with the method described in Gahéry-Ségard et al., 2000 (11).

as regards the use of multimeric complexes and in particular of tetrameric complexes:

a biological sample, preferably peripheral blood mononuclear cells (PBMCs), is brought into contact with tetrameric complexes produced from complexes of Nef peptide as defined above—soluble and biotinylated class II HLA molecule as defined above, and

the labelled cells are analyzed by flow cytometry.

Advantageously, prior to bringing the biological sample into contact with said complex, it is enriched in CD4+ T cells by bringing it into contact with anti-CD4 antibodies, so as to enrich said sample.

The tetramers are prepared as specified, for example, in E. J. Novak et al. (J. Clin. Investig., 1999, 104, R63-R67) or in M. J. Kuroda et al. (J. Virol., 2000, 74, 18, 8751-8756).

Briefly, the tetramers are produced by incubating soluble and biotinylated HLA II molecules with a 10-fold excess of Nef peptides identified and selected in accordance with the invention, for 72 hours at 37° C. and in a 10 mM phosphate citrate buffer containing 0.15 M NaCl, at a pH of between 4.5 and 7.

The tetramerized form is obtained by adding to the preparation streptavidin labelled with a fluorochrome, in an amount four times less (mole for mole) than HLA II molecules. The mixture is incubated overnight at ambient temperature.

To use these tetramers, a suspension of cells (PBMCs, PBMCs depleted of CD8+ cells, T lymphocytes pre-enriched via a step of culturing in vitro with the Nef peptides selected in accordance with the present invention or cloned T lymphocytes) is brought into contact with one or more tetramers (10 to 20 mg/ml) for 1 to 3 hours. After washing, the suspension is analyzed by flow cytometry: the labelling of the cells with the tetramers is visualized by virtue of the fact that these constructs are fluorescent.

The flow cytometry makes it possible to separate the cells labelled with the tetramers from the unlabelled cells and to thus perform cell sorting.

A subject of the present invention is thus also a method for sorting HIV-specific T lymphocytes, characterized in that it comprises at least the following steps:

incubating or bringing a suspension of cells to be sorted into contact, for 1 to 3 hours, with one or more tetramers formed from complexes of Nef peptide as defined above/soluble and biotinylated HLA II molecule, and conjugated to streptavidin labelled with a fluorochrome,

analyzing by flow cytometry, and

sorting the cells labelled with the tetramers.

### EXAMPLE 1

### Principle of the Binding Assays

Peptide Synthesis

The peptides chosen cover the sequence of the Nef protein of the Bru strain published by Wain-Hobson et al. (13). All the peptides were synthesized according to the Fmoc strategy in parallel synthesis on solid phase, purified by HPLC and controlled by mass spectrometry (ES-MS).

Purification of the HLA-DR Molecules

The HLA-DR molecules are purified from various homozygous EBV lines (14) by immunoaffinity. Use may in particular be made of the method described in Southwood et al. (14). Their origin and the various alleles which characterize them are described in table IV.

TABLE IV

| | | | Other DRB |
|---|---|---|---|
| Lines | Specificities | DRB1 alleles | alleles |
| LG2 (14) | HLA-DR1 | DRB1*0101 | — |
| HOM2 | | | |
| SCHU | HLA-DR2 | DRB1*1501 | DRB5*0101 |
| MAT (14) | HLA-DR3 | DRB1*0301 | DRB3*0101 |
| STEILIN | | | |
| BOLETH | HLA-DR4 | DRB1*0401 | DRB4*0101 |
| PREISS (14) | | | |
| PITOUT (14) | HLA-DR7 | DRB1*0701 | DRB4*0101 |
| SWEIG (14) | HLA-DR11 | DRB1*1101 | DRB3*0202 |
| HHKB (17) | HLA-DR13 | DRB1*1301 | DRB3*0101 |

The monomorphic antibody specific for HLA-DR molecules is in particular that described in Southwood et al. (14) or that described in Posch et al. (15). The antibodies are purified from culture supernatants on protein A-Sepharose columns. These antibodies are coupled onto Sepharose 4B or protein A-Sepharose columns for purifying the HLA-DR molecules.

HLA-DR/Peptide Binding Assays

The assays for binding of the peptides to the HLA-DR molecules are competition assays with immuno-enzymatic revelation, initially developed by Hill on the HLA-DR molecule (16). They are carried out in 96-well plates, which makes it possible to study many samples in the same experiment. Briefly, the purified HLA-DR molecules are incubated with a biotinylated peptide which serves as a tracer, and various concentrations of the test peptide.

After 24 to 72 hours of incubation, the samples are neutralized, and then 100 μl of each sample are transferred onto an ELISA plate precoated with the monomorphic antibody specific for HLA-DR molecules. The HLA-DR molecule/biotinylated peptide complexes attached to the bottom of the plate via the monomorphic antibody specific for HLA-DR molecules are revealed by means of a streptavidin-phosphatase conjugate and a fluorescent substrate. The activity of each peptide is characterized by the concentration of this peptide which inhibits 50% of the binding of the biotinylated peptide (IC₅₀).

Choice and Optimization of the Binding Assays

Choice of Alleles (1st Gene)

The alleles studied are all alleles of the French population, the frequency of which exceeds 5% of the population.

They are the alleles DRB1*0101, DRB1*0301, DRB1*0401, DRB1*0701, DRB1*1101, DRB1*1301 and DRB1*1501 (table I). They represent, on their own, 53 to 82% of the alleles of Caucasian populations and are part of various specificities of the HLA-DR series.

Choice of Alleles (2nd Gene)

The alleles studied are the most commonly encountered alleles. They are the alleles HLA-DRB3*0101, HLA-DRB4*0101 and HLA-DRB5*0101.

Assay Specificity

The choice of the biotinylated peptides is the element which determines the specificity of the assay. Most of the cells used have two different HLA-DR molecules (encoded by two alleles) which are both purified by a monomorphic antibody specific for HLA-DR molecules and both recognized by the same antibody. In order to unambiguously study the binding of a peptide to the DRB1 allele, it is necessary to be sure that the biotinylated peptide binds this allele and does not bind the product of the other allele.

For this purpose, the peptides as defined as reagents R1 above were used.

Assay Conditions and Sensitivity

For each HLA-DRB1 molecule, the concentration of MHC II molecules, the concentration of the biotinylated peptide, the incubation pH and the incubation time were optimized as specified in table V below.

TABLE V

| | Protein | | Tracer | | |
|---|---|---|---|---|---|
| | concen- | | concen- | | |
| | tration | | tration | Optimum | Incubation |
| Alleles | (μg/ml) | Tracers | (nM) | pH | time (h) |
| DRB1*0101 | 0.6 | HA 306-318 | 10 | 6 | 24 |
| DRB1*0301 | 2.3 | MT 2-16 | 50 | 4.5 | 72 |
| DRB1*0401 | 1.6 | HA 306-318 | 30 | 6 | 24 |
| DRB1*0701 | 0.4 | YKL | 10 | 5 | 24 |
| DRB1*1101 | 1.3 | HA 306-318 | 20 | 5 | 24 |
| DRB1*1301 | 0.7 | B1 21-36 | 200 | 4.5 | 72 |
| DRB1*1501 | 0.5 | A3 152-166 | 10 | 4.5 | 24 |

The sensitivity of each assay is reflected by the IC₅₀ values observed with the nonbiotinylated peptides which correspond to the tracers, and the results obtained are illustrated in table VI below.

TABLE VI

| | | | | |
|---|---|---|---|---|
| | | Biotinylated | | IC₅₀ |
| Alleles | Frequency | peptides | Sequences | (nM) |

| | | | | |
|---|---|---|---|---|
| DRB1*0101 | 9.3 | HA 306-318 | PKYVKQNTLKLAT | 31 |
| DRB1*0401 | 5.6 | HA 306-318 | PKYVKQNTLKLAT | 44 |
| DRB1*1101 | 9.2 | HA 306-318 | PKYVKQNTLKLAT | 38 |
| DRB1*0701 | 14.0 | YKL | AAYAAAKAAALAA | 34 |
| DRB1*0301 | 10.9 | MT 2-16 | AKTIAYDEEARRGLE | 100 |
| DRB1*1301 | 6.0 | B1 21-36 | TERVRLVTRHIYNREE | 330 |
| DRB1*1501 | 8.0 | A3 152-166 | EAEQLRRAYLDGTGVE | 14 |
| DRB5*0101 | 7.9 | HA 306-318 | PKYVKQNTLKLAT | 6.5 |
| DRB3*0101 | 9.2 | Lol 191-210 | ESWGAVWRIDTPDKLTGPFT | 5 |
| DRB4*0101 | 28.4 | E2/E168 | AGDLLAIETDKATI | 2 |

The frequencies indicated are the allelic frequencies in France and are representative of those of the Caucasian population. They are derived from Colombani (4).

Table VII below illustrates the binding activity for the peptides according to the invention, measured under the conditions specified above:

TABLE VII
Binding activities for the selected NEF
peptides with respect to the HLA-DR molecules which are
predominant in the Caucasian population

| | DR1 | DR3 | DR4 | DR7 | DR11 | DR13 | DR15 | B3 | B5 | B4 |
|---|---|---|---|---|---|---|---|---|---|---|
| Nef1 (1-36) | 700 | >10000 | 250 | 40 | 550 | 325 | 40 | >10000 | 400 | 2500 |
| Nef1.1 (3-17) | 1250 | >10000 | 700 | 48 | 1750 | 3000 | 150 | >10000 | >10000 | >10000 |
| Nef1.2 (8-22) | >10000 | >10000 | >10000 | >10000 | 7500 | 700 | 200 | >10000 | >10000 | >10000 |
| Nef1.3 (11-25) | >10000 | >10000 | >10000 | >10000 | >10000 | 700 | >10000 | >10000 | >10000 | >10000 |
| Nef1.4 (14-28) | 1400 | >10000 | >10000 | >10000 | 1250 | 750 | >10000 | >10000 | >10000 | >10000 |
| Nef1.5 (18-32) | 2750 | >10000 | >10000 | >10000 | >10000 | >10000 | >10000 | >10000 | >10000 | >10000 |
| Nef2 (25-39) | >10000 | 4250 | >10000 | >10000 | >10000 | >10000 | >10000 | >10000 | >10000 | >10000 |
| Nef3 (37-71) | 2500 | >10000 | 70 | 3000 | 2000 | >10000 | >10000 | >10000 | >10000 | 8000 |
| Nef4 (66-94) | 55 | >10000 | 45 | 5000 | 850 | >10000 | 750 | >10000 | 12 | 225 |
| Nef4.1 (66-80) | 1500 | >10000 | >10000 | >10000 | >10000 | >10000 | >10000 | >10000 | >10000 | >10000 |
| Nef4.2 (68-82) | 250 | >10000 | >10000 | >10000 | >10000 | >10000 | 7000 | >10000 | >10000 | >10000 |
| Nef4.3 (72-86) | 2000 | >10000 | 2000 | >10000 | 4000 | >10000 | 1930 | >10000 | 1500 | 2430 |
| Nef4.4 (74-88) | 350 | >10000 | 225 | >10000 | 850 | >10000 | 667 | >10000 | 700 | 550 |
| Nef4.5 (77-91) | 12 | >10000 | 60 | >10000 | 700 | >10000 | 1330 | >10000 | 28 | 300 |
| Nef4.6 (79-93) | 55 | >10000 | 150 | >10000 | 2750 | >10000 | >10000 | >10000 | 45 | >10000 |
| Nef4.7 (83-97) | 500 | >10000 | >10000 | >10000 | 7500 | >10000 | >10000 | >10000 | 3250 | >10000 |
| Nef5 (86-100) | 6000 | >10000 | >10000 | >10000 | >10000 | >10000 | >10000 | >10000 | 8000 | >10000 |
| Nef6 (100-114) | >10000 | >10000 | >10000 | >10000 | 3000 | >10000 | 1750 | >10000 | >10000 | 1750 |
| Nef7 (113-128) | >10000 | >10000 | >10000 | 4000 | >10000 | >10000 | 9000 | 75 | >10000 | >10000 |
| Nef8 (117-132) | >10000 | >10000 | 8000 | >10000 | >10000 | >10000 | >10000 | 120 | >10000 | >10000 |
| Nef9 (132-147) | 8.5 | >10000 | >10000 | 5500 | 500 | >10000 | 1500 | >10000 | 8000 | 500 |
| Nef10 (137-168) | 650 | 175 | 150 | 65 | 45 | >10000 | 1250 | 3500 | 250 | 55 |
| Nef10.1 (137-151) | 150 | >10000 | 2000 | 650 | 95 | >10000 | >10000 | >10000 | 4500 | 2200 |
| Nef10.2 (139-153) | 85 | >10000 | 250 | 120 | 250 | >10000 | >10000 | >10000 | 900 | 605 |
| Nef10.3 (141-155) | 850 | >10000 | 250 | 250 | 350 | >10000 | >10000 | >10000 | 1500 | 1030 |
| Nef10.4 (143-157) | 9000 | 1470 | 2750 | 2250 | >10000 | >10000 | >10000 | >10000 | >10000 | 1730 |
| Nef10.5 (146-160) | 550 | 2500 | 2000 | >10000 | >10000 | >10000 | >10000 | >10000 | >10000 | >10000 |
| Nef10.6 (151-165) | >10000 | >10000 | >10000 | >10000 | >10000 | >10000 | >10000 | >10000 | 1500 | >10000 |
| Nef11 (155-185) | 2000 | >10000 | >10000 | 3750 | 3000 | >10000 | 50 | >10000 | 3000 | 450 |
| Nef12 (175-190) | >10000 | 3500 | 2500 | 700 | 125 | >10000 | 13 | 145 | >10000 | 275 |
| Nef13 (182-198) | 5000 | 55 | 3250 | 150 | 48 | 750 | >10000 | 250 | 10 | 6330 |
| Nef13.1 (178-192) | >10000 | 125 | >10000 | 350 | 8 | 867 | >10000 | 28 | 550 | >10000 |
| Nef13.2 (181-195) | >10000 | 73 | >10000 | 933 | 18 | 350 | >10000 | 60 | 6 | >10000 |
| Nef13.3 (183-197) | >10000 | 567 | >10000 | 900 | 11 | 250 | >10000 | 5000 | 15 | >10000 |
| Nef13.4 (187-201) | >10000 | >10000 | >10000 | 225 | 6 | 350 | >10000 | >10000 | 300 | >10000 |
| Nef13.5 (189-203) | >10000 | 1470 | >10000 | 250 | 8 | 600 | >10000 | >10000 | 600 | >10000 |
| Nef13.6 (191-205) | >10000 | 700 | >10000 | 2500 | 550 | 3000 | >10000 | >10000 | 8000 | >10000 |

The results are expressed in the form of concentrations giving 50% inhibition of maximum binding. The unit is nM.

### EXAMPLE 2

### Proliferation Assay

To verify the stimulation of the CD4+ T cell proliferation, using an immunogenic composition according to the invention, a proliferation assay is carried out in vitro.

Cells (PBMCs) extracted from peripheral blood were cultured in 96-well microplates in a proportion of 5×10⁵ cells per well, in a final volume of 200 μl of complete medium. The cells were stimulated with 20 μg/ml of a mixture of peptides according to the invention, or were not stimulated. After 72 hours of culturing at 37° C., the cells were incubated overnight with 0.25 μCi of [³H] thymidine (Amersham, Life technologie). The cells were recovered and the incorporation of [³H] thymidine was measured in the cellular DNA.

Stimulation of the CD4+ T cells was effectively observed.

Other cells can be used: PBMCs depleted of CD8+ cells, T lymphocytes pre-enriched via a step of culturing in vitro with the peptides as defined above or cloned lymphocytes.

Briefly, the enrichment protocol is as follows:

PMBCs, separated on a ficoll gradient, are cultured at 37° C. in the presence of 0.1 to 10 mg/ml of peptides, in RPMI medium supplemented with 10% of human serum. On the 7th and 11th day of culturing, 50 units of recombinant human IL-2 are added to the culture. The cells are harvested on the 14th day.

### EXAMPLE 3

### Elispot

The ELISPOT makes it possible to detect the cells specific for a peptide and secreting a given cytokine.

50 μl/well of murine anti-human γ-IFN antibody diluted in PBS buffer at a concentration of 4 μg/ml are incubated in nitrocellulose-bottomed 96-well plates overnight at 4° C. in a humid chamber.

The wells are washed with PBS and saturated with RPMI medium containing 10% of calf serum, for 2 hours at 37° C.

If needed, suitable presenting cells, such as autologous or heterologous PBMCs, lymphoblastoid cells obtained after infection with the EBV virus or genetically modified cells, are used and are distributed into the wells. The Nef peptides as defined in the invention are then added at various concentrations (10, 5 and 1 μg/ml).

The effector cells (PBMCs, PBMCs depleted of CD8+ cells, T lymphocytes pre-enriched via a step of culturing in vitro with the Nef peptides or cloned lymphocytes) are added to the 96-well plates in a proportion of 20 000 cells/well.

The culture is incubated for 24 hours at 37° C. in an atmosphere containing 5% CO₂.

The plates are then washed and incubated for 2 hours with 100 μl of a rabbit antiserum specific for human γ-IFN.

After washing, an anti-rabbit IgG antibody conjugated to biotin and then streptavidin conjugated to alkaline phosphatase are successively added for 1 hour.

Finally, the spots are revealed using a chromogenic substrate for alkaline phosphatase. The spots are counted under a microscope. Negative controls are given by the well containing no peptides. The positive controls are provided by the wells containing mitogenic agents such as ionomycin (500 ng/ml) and phytohemagglutinin (PHA) (10 μg/ml).

Bibliographic References

1. E. S. Rosenberg et al., Science, 1997, 278, 1447.

2. S. A. Kalams et al., J. Virol., 1999, 73, 6715.

3. B. Autran et al., Science, 1997, 277, 112.

4. J. Colombani, HLA: fonctions immunitaires et applications médicales [Immune functions and medical applications], Eds. John Libbey Eurotext, 1993.

5. J. Choppin et al., J. Immunol., 1991, 147, 569.

6. J. Choppin et al., J. Immunol., 1991, 147, 575.

7. M. Dalod et al., J. Clin. Invest., 1999, 104, 1431.

8. J. Estaquier et al., Mol. Immunol., 1992, 29, 489.

9. J. D. Ahlers et al., Proc. Natl. Acad. Sci. USA, 1997, 94, 10856.

10. P. A. Wentworth et al., Vaccine, 1994, 12, 117.

11. H. Gahery-Segard et al., J. Virol., 2000, 74, 1694.

12. A. Takahashi et al., Vaccine, 1998, 16, 1537.

13. S. Wain-Hobson et al., Cell, 1985, 40, 9.

14. S. Southwood et al., J. Immunol., 1998, 160, 3363-3373.

15. P. E. Posch et al., Eur. J. Immunol., 1996, 26, 1884.

16. C. M. Hill et al., J. Immunol., 1994, 152, 2890.

17. M. P. Davenport et al., Proc. Natl. Acad. Sci. USA, USA, 1995, 92, 6567.

1. A mixture of peptides derivated from a HIV Nef protein, characterized in that each one of said peptides binds to at least three HLA-DR molecules encoded by the alleles selected from the group consisting of the HLA alleles DRB1*0101, DRB1*0301, DRB1*0401, DRB1*0701, DRB1*1101, DRB1*1301 and DRB1*1501 (molecules DR1, DR3, DR4, DR7, DR11, DR13 and DR15) and to at least one HLA-DR molecule encoded by the alleles selected from the group consisting of the HLA alleles DRB3*0101, DRB4*0101 and DRB5*0101 (B3, B4 and B5), with a binding activity<1 000 nM, said mixture of peptides binding all the abovementioned alleles.

2. The mixture of peptides as claimed in claim 1, characterized in that said peptides originate from the Nef protein of any HIV-1 strain.

3. The mixture of peptides as claimed in claim 1 or claim 2, characterized in that said peptides are selected from the group consisting of the peptide corresponding to positions 1-36 (Nef1 peptide), the peptide corresponding to positions 66-94 (Nef4 peptide), the peptide corresponding to positions 74-88 (Nef4.4 peptide), the peptide corresponding to positions 77-91 (Nef4.5 peptide), the peptide corresponding to positions 137-168 (Nef10 peptide), the peptide corresponding to positions 139-153 (Nef10.2 peptide), the peptide corresponding to positions 175-190 (Nef12 peptide), the peptide corresponding to positions 182-198 (Nef13 peptide), the peptide corresponding to positions 178-192 (Nef13.1 peptide), the peptide corresponding to positions 181-195 (Nef13.2 peptide), the peptide corresponding to positions 183-197 (Nef13.3 peptide), the peptide corresponding to positions 187-201 (Nef13.4 peptide) and the peptide corresponding to positions 189-203 (Nef13.5 peptide) of the Nef protein, with reference to the Bru strain (13).

4. The mixture of peptides as claimed in claim 3, characterized in that it is selected from the group consisting of the following mixtures:
a mixture comprising: the peptide corresponding to positions 66-94 (Nef4), the peptide corresponding to positions 182-198 (Nef13);
a mixture comprising: the peptide corresponding to positions 66-94 (Nef4), the peptide corresponding to positions 178-192 (Nef13.1);
a mixture comprising: the peptide corresponding to positions 66-94 (Nef4), the peptide corresponding to positions 181-195 (Nef13.2);
a mixture comprising: the peptide corresponding to positions 74-88 (Nef4.4), the peptide corresponding to positions 182-198 (Nef13);
a mixture comprising: the peptide corresponding to positions 74-88 (Nef4.4), the peptide corresponding to positions 178-192 (Nef13.1);
a mixture comprising: the peptide corresponding to positions 74-88 (Nef4.4), the peptide corresponding to positions 181-195 (Nef13.2);
a mixture comprising: the peptide corresponding to positions 1-36 (Nef1), the peptide corresponding to positions 66-94 (Nef4) and the peptide corresponding to positions 182-198 (Nef13);
a mixture comprising: the peptide corresponding to positions 1-36 (Nef1), the peptide corresponding to positions 74-88 (Nef4.4) and the peptide corresponding to positions 182-198 (Nef13);
a mixture comprising: the peptide corresponding to positions 1-36 (Nef1), the peptide corresponding to positions 77-91 (Nef4.5) and the peptide corresponding to positions 182-198 (Nef13);
a mixture comprising: the peptide corresponding to positions 1-36 (Nef1), the peptide corresponding to positions 66-94 (Nef4) and the peptide corresponding to positions 178-192 (Nef13.1);
a mixture comprising: the peptide corresponding to positions 1-36 (Nef1), the peptide corresponding to positions 74-88 (Nef4.4) and the peptide corresponding to positions 178-192 (Nef13.1);
a mixture comprising: the peptide corresponding to positions 1-36 (Nef1), the peptide corresponding to positions 77-91 (Nef4.5) and the peptide corresponding to positions 178-192 (Nef13.1);
a mixture comprising: the peptide corresponding to positions 1-36 (Nef1), the peptide corresponding to positions 66-94 (Nef4) and the peptide corresponding to positions 181-195 (Nef13.2);
a mixture comprising: the peptide corresponding to positions 1-36 (Nef1), the peptide corresponding to positions 74-88 (Nef4.4) and the peptide corresponding to positions 181-195 (Nef13.2);
a mixture comprising: the peptide corresponding to positions 1-36 (Nef1), the peptide corresponding to positions 77-91 (Nef4.5) and the peptide corresponding to positions 181-195 (Nef13.2);
a mixture comprising: the peptide corresponding to positions 1-36 (Nef1), the peptide corresponding to positions 137-168 (Nef10 ) and the peptide corresponding to positions 175-190 (Nef12);
a mixture comprising: the peptide corresponding to positions 1-36 (Nef1), the peptide corresponding to positions 137-168 (Nef10) and the peptide corresponding to positions 182-198 (Nef13);
a mixture comprising: the peptide corresponding to positions 1-36 (Nef1), the peptide corresponding to positions 137-168 (Nef10) and the peptide corresponding to positions 178-192 (Nef13.1);
a mixture comprising: the peptide corresponding to positions 1-36 (Nef1), the peptide corresponding to positions 137-168 (Nef10) and the peptide corresponding to positions 181-195 (Nef13.2);
a mixture comprising: the peptide corresponding to positions 1-36 (Nef1), the peptide corresponding to positions 139-153 (Nef10.2) and the peptide corresponding to positions 182-198 (Nef13);
a mixture comprising: the peptide corresponding to positions 1-36 (Nef1), the peptide corresponding to positions 139-153 (Nef10.2) and the peptide corresponding to positions 178-192 (Nef13.1);
a mixture comprising: the peptide corresponding to positions 1-36 (Nef1), the peptide corresponding to positions 139-153 (Nef10.2) and the peptide corresponding to positions 181-195 (Nef13.2);
a mixture comprising: the peptide corresponding to positions 1-36 (Nef1), the peptide corresponding to positions 175-190 (Nef12) and the peptide corresponding to positions 182-198 (Nef13);
a mixture comprising: the peptide corresponding to positions 1-36 (Nef1), the peptide corresponding to positions 175-190 (Nef12) and the peptide corresponding to positions 178-192 (Nef13.1);
a mixture comprising: the peptide corresponding to positions 1-36 (Nef1), the peptide corresponding to positions 175-190 (Nef12) and the peptide corresponding to positions 181-195 (Nef13.2);
a mixture comprising: the peptide corresponding to positions 66-94 (Nef4), the peptide corresponding to positions 137-168 (Nef10) and the peptide corresponding to positions 182-198 (Nef13);
a mixture comprising: the peptide corresponding to positions 66-94 (Nef4), the peptide corresponding to positions 137-168 (Nef10) and the peptide corresponding to positions 178-192 (Nef13.1);
a mixture comprising: the peptide corresponding to positions 66-94 (Nef4), the peptide corresponding to positions 137-168 (Nef10) and the peptide corresponding to positions 181-195 (Nef13.2);
a mixture comprising: the peptide corresponding to positions 66-94 (Nef4), the peptide corresponding to positions 139-153 (Nef10.2) and the peptide corresponding to positions 182-198 (Nef13);
a mixture comprising: the peptide corresponding to positions 66-94 (Nef4), the peptide corresponding to positions 139-153 (Nef10.2) and the peptide corresponding to positions 178-192 (Nef13.1);
a mixture comprising: the peptide corresponding to positions 66-94 (Nef4), the peptide corresponding to positions 139-153 (Nef10.2) and the peptide corresponding to positions 181-195 (Nef13.2);
a mixture comprising: the peptide corresponding to positions 66-94 (Nef4), the peptide corresponding to positions 175-190 (Nef12) and the peptide corresponding to positions 182-198 (Nef13);
a mixture comprising: the peptide corresponding to positions 66-94 (Nef4), the peptide corresponding to positions 175-190 (Nef12) and the peptide corresponding to positions 178-192 (Nef13.1);
a mixture comprising: the peptide corresponding to positions 66-94 (Nef4), the peptide corresponding to positions 175-190 (Nef12) and the peptide corresponding to positions 181-195 (Nef13.2);
a mixture comprising: the peptide corresponding to positions 66-94 (Nef4), the peptide corresponding to positions 175-190 (Nef12) and the peptide corresponding to positions 183-197 (Nef13.3);
a mixture comprising: the peptide corresponding to positions 66-94 (Nef4), the peptide corresponding to positions 175-190 (Nef12) and the peptide corresponding to positions 187-201 (Nef13.4);
a mixture comprising: the peptide corresponding to positions 66-94 (Nef4), the peptide corresponding to positions 175-190 (Nef12) and the peptide corresponding to positions 189-203 (Nef13.5);
a mixture comprising: the peptide corresponding to positions 74-88 (Nef4.4), the peptide corresponding to positions 137-168 (Nef10) and the peptide corresponding to positions 182-198 (Nef13);
a mixture comprising: the peptide corresponding to positions 74-88 (Nef4.4), the peptide corresponding to positions 137-168 (Nef10) and the peptide corresponding to positions 178-192 (Nef13.1);
a mixture comprising: the peptide corresponding to positions 74-88 (Nef4.4), the peptide corresponding to positions 137-168 (Nef10) and the peptide corresponding to positions 181-195 (Nef13.2);
a mixture comprising: the peptide corresponding to positions 74-88 (Nef4.4), the peptide corresponding to positions 175-190 (Nef12) and the peptide corresponding to positions 182-198 (Nef13);
a mixture comprising: the peptide corresponding to positions 74-88 (Nef4.4), the peptide corresponding to positions 175-190 (Nef12) and the peptide corresponding to positions 178-192 (Nef13.1);
a mixture comprising: the peptide corresponding to positions 74-88 (Nef4.4), the peptide corresponding to positions 175-190 (Nef12) and the peptide corresponding to positions 181-195 (Nef13.2);
a mixture comprising: the peptide corresponding to positions 74-88 (Nef4.4), the peptide corresponding to positions 175-190 (Nef12) and the peptide corresponding to positions 183-197 (Nef13.3);
a mixture comprising: the peptide corresponding to positions 77-91 (Nef4.5), the peptide corresponding to positions 175-190 (Nef12) and the peptide corresponding to positions 182-198 (Nef13);
a mixture comprising: the peptide corresponding to positions 77-91 (Nef4.5), the peptide corresponding to positions 175-190 (Nef12) and the peptide corresponding to positions 178-192 (Nef13.1);
a mixture comprising: the peptide corresponding to positions 77-91 (Nef4.5), the peptide corresponding to positions 175-190 (Nef12) and the peptide corresponding to positions 181-195 (Nef13.2);
a mixture comprising: the peptide corresponding to positions 77-91 (Nef4.5), the peptide corresponding to positions 175-190 (Nef12) and the peptide corresponding to positions 183-197 (Nef13.3);
a mixture comprising: the peptide corresponding to positions 137-168 (Nef10), the peptide corresponding to positions 175-190 (Nef12) and the peptide corresponding to positions 182-198 (Nef13);
a mixture comprising: the peptide corresponding to positions 137-168 (Nef10), the peptide corresponding to positions 175-190 (Nef12) and the peptide corresponding to positions 178-192 (Nef13.1);
a mixture comprising: the peptide corresponding to positions 137-168 (Nef10), the peptide corresponding to positions 175-190 (Nef12) and the peptide corresponding to positions 181-195 (Nef13.2);
a mixture comprising: the peptide corresponding to positions 137-168 (Nef10), the peptide corresponding to positions 175-190 (Nef12) and the peptide corresponding to positions 183-197 (Nef13.3);
a mixture comprising: the peptide corresponding to positions 137-168 (Nef10), the peptide corresponding to positions 175-190 (Nef12) and the peptide corresponding to positions 187-201 (Nef13.4);
a mixture comprising: the peptide corresponding to positions 137-168 (Nef10), the peptide corresponding to positions 175-190 (Nef12) and the peptide corresponding to positions 189-203 (Nef13.5).

5. An immunogenic composition, characterized in that it comprises at least one mixture of peptides as claimed in any one of claims 1 to 4, combined with at least one pharmaceutically acceptable vehicle and, optionally, with at least one adjuvant.

6. The immunogenic composition as claimed in claim 5, characterized in that said peptides are either in the form of lipopeptides, or are incorporated into a recombinant virus or a viral vector for gene therapy, or are included in a protein, or are chemically modified.

7. The immunogenic composition as claimed in claim 5 or claim 6, characterized in that said mixture of peptides is combined:
with one or more peptides or lipopeptides containing one or more CD8+ epitopes, and/or
with other peptides containing a CD4+ epitope, which peptides bind at least to one HLA-DR molecule encoded by the alleles selected from the group consisting of the HLA alleles DRB1*0101, DRB1*0301, DRB1*0401, DRB1*0701, DRB1*1101, DRB1*1301 and DRB1*1501 (molecules DR1, DR3, DR4, DR7, DR11, DR13 and DR15) or to at least one HLA-DR molecule encoded by the alleles selected from the group consisting of the HLA alleles DRB3*0101, DRB4*0101 and DRB5*0101 (B3, B4 and B5), with a binding activity<1 000 nM, and/or
with other peptides comprising multiple CD4+ epitopes, and/or
with one or more peptides or lipopeptides containing one or more B epitopes recognized specifically by antibodies directed against said epitopes.

8. The immunogenic composition as claimed in claim 7, characterized in that the peptides containing a CD4+ epitope are selected from the group consisting of the peptides corresponding to positions 37-71 (Nef3), to positions 113-128 (Nef7), to positions 117-132 (Nef8), to positions 132-147 (Nef9) and to positions 155-185 (Nef11) of the HIV-1 Nef protein.

9. An immunogenic composition, characterized in that it advantageously comprises the sequences encoding the peptides as defined in claims 1 to 4.

10. An anti-HIV vaccine, characterized in that it includes an immunogenic composition as claimed in any one of claims 5 to 9.

11. A peptide derivated from a HIV Nef protein, characterized:
in that it contains a CD4+ epitope capable of having a binding activity<1 000 nM with respect to at least one HLA II molecule (HLA-DR) which is predominant in Caucasian populations (1st gene and/or 2nd gene) and of being recognized by CD4+ T lymphocytes specific for said peptides, and
in that it is selected from the group consisting of the following fragments of Nef protein, with reference to the Bru strain: the fragment corresponding to positions 1-36 of the Nef protein (Nef1), the fragment corresponding to positions 3-17 (Nef1.1), the fragment corresponding to positions 8-22 (Nef1.2), the fragment corresponding to positions 11-25 (Nef1.3), the fragment corresponding to positions 14-28 (Nef1.4), the fragment corresponding to positions 37-71 of the Nef protein (Nef3), the fragment corresponding to positions 66-94 (Nef4), the fragment corresponding to positions 68-82 (Nef4.1), the fragment corresponding to positions 74-88 (Nef4.4), the fragment corresponding to positions 77-91 (Nef4.5), the fragment corresponding to positions 79-93 (Nef4.6), the fragment corresponding to positions 83-97 (Nef4.7), the fragment corresponding to positions 113-128 (Nef7), the fragment corresponding to positions 117-132 (Nef8), the fragment corresponding to positions 132-147 (Nef9), the fragment corresponding to positions 137-168 (Nef10), the fragment corresponding to positions 137-151 (Nef10.1), the fragment corresponding to positions 139-153 (Nef10.2), the fragment corresponding to positions 141-155 (Nef10.3), the fragment corresponding to positions 146-160 (Nef10.5), the fragment corresponding to positions 155-185 (Nef11), the fragment corresponding to positions 175-190 (Nef12), the fragment corresponding to positions 182-198 (Nef13), the fragment corresponding to positions 178-192 (Nef13.1), the fragment corresponding to positions 181-195 (Nef13.2), the fragment corresponding to positions 183-197 (Nef13.3), the fragment corresponding to positions 187-201 (Nef13.4), the fragment corresponding to positions 189-203 (Nef13.5), and the fragment corresponding to positions 191-205 (Nef13.6).

12. A diagnostic reagent, characterized in that it is selected from the group consisting of a mixture of peptides as claimed in any one of claims 1 to 4 or one of the peptides as claimed in claim 11, said peptides being optionally labelled or complexed, in the form of multimeric complexes.

13. A method for evaluating the immune state of an individual, characterized in that it comprises a step of detecting the presence of CD4+ T cells specific for the Nef peptides as defined in claims 1 to 4 or in claim 11, using an ELISPOT assay, a T cell proliferation assay or an assay using multimeric complexes.

14. A method for sorting HIV-specific T lymphocytes, characterized in that it comprises at least the following steps:
incubating or bringing a suspension of cells to be sorted into contact, for 1 to 3 hours, with one or more tetramers formed from complexes of Nef peptide as defined in claims 1 to 4 or in claim 11/soluble and biotinylated HLA II molecule, and conjugated to streptavidin labeled with a fluorochrome,
analyzing by flow cytometry, and
sorting the cells labeled with the tetramers.


SEQUENCE LISTING (7 sequences)

SEQ ID NO: 1 - protein, 13 aa, artificial
  immunogenic peptide
PKYVKQNTLKLAT

SEQ ID NO: 2 - protein, 15 aa, artificial
  immunogenic peptide
EAEQLRAYLDGTGVE

SEQ ID NO: 3 - protein, 15 aa, artificial
  immunogenic peptide
AKTIAYDEEARRGLE

SEQ ID NO: 4 - protein, 13 aa, artificial
  immunogenic peptide
AAYAAAKAAALAA

SEQ ID NO: 5 - protein, 16 aa, artificial
  immunogenic peptide
TERVRLVTRHIYNREE

SEQ ID NO: 6 - protein, 20 aa, artificial
  immunogenic peptide
ESWGAVWRIDTPDKLTGPFT

SEQ ID NO: 7 - protein, 14 aa, artificial
  immunogenic peptide
AGDLLAIETDKATI